(19) United States
(12) Patent Application Publication
(10) Pub. No.: US 2024/0374876 A1
(43) Pub. Date: Nov. 14, 2024

(54) METHOD FOR PRODUCING PARISON FOR BALLOON CATHETER, METHOD FOR PRODUCING BALLOON CATHETER, AND DEVICE FOR PRODUCING PARISON FOR BALLOON CATHETER

(71) Applicant: TERUMO KABUSHIKI KAISHA, Tokyo (JP)

(72) Inventors: Yuuji YAMADA, Kanagawa (JP); Takuma KATAHIRA, Kanagawa (JP); Shingo ISHII, Kanagawa (JP)

(73) Assignee: TERUMO KABUSHIKI KAISHA, Tokyo (JP)

(21) Appl. No.: 18/658,164

(22) Filed: May 8, 2024

(30) Foreign Application Priority Data
May 11, 2023 (JP) 2023-078676

(51) Int. Cl. A61M 25/10

(52) U.S. Cl. CPC A61M 25/1029; A61M 2207/10

(57) ABSTRACT

A method for producing a parison for a balloon catheter, a method for producing a balloon catheter, and a device for producing a parison for a balloon catheter, by which a shape of the parison can be stably formed by lowering a heating temperature of a tube. A method for producing a parison for a balloon catheter includes: fixing a pair of fixing portions of a thermoplastic tube extending along a major axis direction; heating a pair of heating portions located between the pair of fixing portions; cooling at least a part of a portion between the pair of heating portions of the tube during the heating a pair of heating portions located between the pair of fixing portions; and pulling the tube in a direction in which the pair of fixing portions are separated from each other, and stretching the pair of heating portions.

### CROSS-REFERENCES TO RELATED APPLICATIONS

This application claims priority to Japanese Application No. 2023-078676 filed on May 11, 2023, the entire content of which is incorporated herein by reference.

### TECHNOLOGICAL FIELD

The present disclosure generally relates to a method for producing a parison which is an intermediate product during production of a balloon provided in a balloon catheter, a method for producing a balloon catheter, and a device for producing a parison for a balloon catheter.

### BACKGROUND DISCUSSION

A balloon catheter is widely used to improve a lesion area (stenosed site) occurring in a body lumen. A balloon catheter generally includes an elongated shaft and a balloon provided on a distal side of the shaft and being inflatable in a radial direction. The lesion area can be widened by inflating the deflated balloon after reaching a target location within the body via a narrow body lumen.

Main constituent elements of the balloon catheter are the balloon and the shaft as described above. As for the balloon, a tube formed by extrusion molding or the like is processed into an intermediate product during the production, which is called a parison, and the parison is biaxially stretched in a radial direction and an axial direction, thereby forming the balloon finally. The formed balloon is assembled by being attached to the shaft. International Patent Application Publication No. WO2014/141382 discloses a production method in which a balloon is processed from a tube through a parison.

The processing from the tube to the parison is performed by maintaining an original diameter of a part of the tube in a major axis direction, which is served as a part to be processed into a balloon, and stretching, in the major axis direction, parts on both sides in the major axis direction of the part to be processed into the balloon. In the parison, the part stretched in the major axis direction of the tube is oriented to increase the strength. Therefore, when the parison is biaxially stretched, the part stretched in the major axis direction of the tube is not expanded, and only the part of the tube whose original diameter is maintained is expanded to form the balloon.

When the parison is produced, the parts of the tube on both sides in the major axis direction of the part to be processed into the balloon are heated and stretched in the major axis direction. The heating for the tube is performed by disposing a heating target part so as to be surrounded by a heating surface of a heating unit having a space in the heating unit. However, when the heat is transferred from a portion of the tube which is heated by the heating unit to a portion thereof which is not heated, a boundary between the heated portion and the non-heated portion is ambiguous, and the shape of the formed parison may become unstable. When the shape of the parison is unstable, the shape of the balloon formed from the parison may be influenced.

### SUMMARY

A method for producing a parison for a balloon catheter, a method for producing a balloon catheter, and a device for producing a parison for a balloon catheter are disclosed, by which a temperature difference between a heated portion and a non-heated portion of a tube is clarified and a shape of the parison can be stably formed.

(1) A method for producing a parison for a balloon catheter includes: fixing a pair of fixing portions of a thermoplastic tube extending along a major axis direction, the pair of fixing portions being separated from each other in the major axis direction (a fixing step); heating a pair of heating portions of the tube, the pair of heating portions being located between the pair of fixing portions and separated from each other in the major axis direction (a heating step); cooling at least a part of a portion between the pair of heating portions of the tube during the heating step (a cooling step); and pulling the tube in a direction in which the pair of fixing portions are separated from each other, and stretching the pair of heating portions of the tube heated by the heating step (a stretching step). A thick portion having an outer diameter larger than that of the stretched portion is formed between the pair of heating portions stretched by the stretching step.

(9) A device for producing a parison for a balloon catheter includes: a fixing mechanism configured to fix a pair of fixing portions of a thermoplastic tube extending along a major axis direction, the pair of fixing portions being separated from each other in the major axis direction; a heating mechanism configured to heat a pair of heating portions of the tube, the pair of heating portions being located between the pair of fixing portions and separated from each other in the major axis direction; a cooling mechanism configured to cool a portion between the pair of heating portions of the tube; and a pulling mechanism configured to move the fixing mechanism in a direction in which a distance between the pair of fixing portions of the tube is increased.

In the method for producing a parison for a balloon catheter configured as described above, when the tube is heated, a clear temperature difference can be established between the heating portion and the portion of the tube between the heating portions. Therefore, the shape of the parison can be stably formed in the stretching step.

(2) In the method for producing a parison for a balloon catheter according to the above (1), in the cooling step, at least a part of a cooling unit may come into contact with a surface of the tube. Accordingly, the tube can be stably cooled by heat conduction.

(3) In the method for producing a parison for a balloon catheter according to the above (1), in the cooling step, a cooling unit may cool a space surrounded by a wall surface having a rectangular cross-sectional shape obtained by cutting along a plane perpendicular to the major axis direction. Accordingly, the tube can be stably cooled by combining the radiant heat and the convection heat transfer.

(4) In the method for producing a parison for a balloon catheter according to the above (2) or (3), in the cooling step, a surface of the portion between the pair of heating portions may be maintained at a temperature lower than a glass transition temperature of the tube. Accordingly, the temperature rise in the part of the tube to be the thick portion can be prevented, and the shape of a shoulder portion of the parison to be produced can be clearly formed.

(5) In the method for producing a parison for a balloon catheter according to any one of the above (2) to (4), in the heating step, a surface of the heating portion may be maintained at a temperature higher than the glass transition temperature of the tube. Accordingly, the heating portion of the tube can be reliably stretched in the stretching step to increase the strength.

(6) In the method for producing a parison for a balloon catheter according to any one of the above (1) to (5), a difference between a surface temperature of the tube in the heating step and a surface temperature of the tube in the cooling step can be 50° C. or higher and 140° C. or lower (i.e., 50° C. to 140° C.). Accordingly, the shape of the thick portion of the tube can be stably formed by providing a sufficient temperature difference between the heating portion and the cooling portion of the tube.

(7) In the method for producing a parison for a balloon catheter according to any one of the above (1) to (6), in the cooling step, a time from a start of cooling the tube to a state in which a temperature lower than the glass transition temperature of the tube is maintained may be 5 seconds or longer and 45 seconds or shorter. Accordingly, the non-heated portion of the tube can be reliably cooled in accordance with the heating time of the tube.

(8) A method for producing a balloon catheter includes: biaxially stretching the parison for a balloon catheter, which is produced by the production method according to any one of the above (1) to (7) to form a balloon; and disposing a shaft in the formed balloon. The shape of the parison for a balloon catheter is stably formed by the production method according to any one of the above (1) to (7), and therefore, the balloon catheter including the balloon having a stable shape can be produced.

(10) A method for producing a parison for a balloon catheter, the method comprising: fixing a pair of fixing portions of a tube extending along a major axis direction, the pair of fixing portions being separated from each other in the major axis direction; heating a pair of heating portions of the tube, the pair of heating portions being located between the pair of fixing portions and separated from each other in the major axis direction; cooling at least a part of a portion between the pair of heating portions of the tube during the heating of the pair of heating portions of the tube; and pulling the tube in a direction in which the pair of fixing portions are separated from each other, and stretching the pair of heating portions of the tube heated by the heating of the pair of heating portions of the tube.

In the device for producing a parison for a balloon catheter configured as described above, when the tube is heated, a clear temperature difference can be established between the heating portion and the portion of the tube between the heating portions, and the shape of the parison can be stably formed.

### BRIEF DESCRIPTION OF THE DRAWINGS

FIG. 1 is a front view of an entire balloon catheter.

FIG. 2 is a front view of an entire parison.

FIG. 3 is an enlarged cross-sectional view of a vicinity of a central portion of the parison.

FIG. 4 is a front view of a parison production device and shows a state in which a tube is fixed to a fixing mechanism.

FIG. 5 is a front view of the parison production device, and shows a state in which a heating mechanism and a cooling mechanism are closed.

FIG. 6 is a cross-sectional view showing an opened state of a heating mold in which a tube is disposed.

FIG. 7 is a cross-sectional view showing a closed state of the heating mold in which the tube is disposed.

FIG. 8 is a cross-sectional view showing an opened state of a cooling mold in which the tube is disposed.

FIG. 9 is a cross-sectional view showing a closed state of the cooling mold in which the tube is disposed.

FIG. 10 is a front view of the parison production device and shows a state in which the tube is stretched by a pulling mechanism.

FIG. 11A is an enlarged cross-sectional view of a vicinity of a central portion of the parison before biaxial stretching process.

FIG. 11B is an enlarged cross-sectional view of the vicinity of the central portion of the parison after the biaxial stretching process.

FIG. 12 is an enlarged cross-sectional view of a vicinity of a produced balloon in a state in which a shaft is disposed in the balloon.

FIG. 13 is a cross-sectional view of the heating mold in which a plurality of tubes are disposed.

FIG. 14 is a cross-sectional view of the cooling mold in which a plurality of tubes are arranged.

FIG. 15 is a cross-sectional view showing an opened state of a heating mold according to a modification in which a tube is disposed.

FIG. 16 is a cross-sectional view showing a closed state of the heating mold according to the modification in which the tube is disposed.

FIG. 17 is a cross-sectional view showing an opened state of a cooling mold according to the modification in which a tube is disposed.

FIG. 18 is a cross-sectional view showing a closed state of the cooling mold according to the modification in which the tube is disposed.

FIG. 19 is a cross-sectional view of a closed state of the heating mold according to the modification in which a plurality of tubes are disposed.

FIG. 20 is a cross-sectional view of a closed state of the cooling mold according to the modification in which the plurality of tubes are disposed.

FIG. 21 is a cross-sectional view of a closed state of a heating mold according to a second modification in which a tube is disposed.

FIG. 22 is a cross-sectional view of a closed state of a cooling mold according to the second modification in which the tube is disposed.

### DETAILED DESCRIPTION

Set forth below with reference to the accompanying drawings is a detailed description of embodiments of a method for producing a parison which is an intermediate product during production of a balloon provided in a balloon catheter, a method for producing a balloon catheter, and a device for producing a parison for a balloon catheter. Note that a dimensional ratio in the drawings may be exaggerated for convenience of illustration and may be different from an actual ratio. In the present specification, a side of a balloon catheter 10 to be inserted into a body lumen is referred to as “distal end” or “distal side”, and a hand-side to be operated is referred to as “proximal end” or “proximal side”.

In a method for producing a parison for a balloon catheter according to an embodiment of the present disclosure, a part of a tube 30 formed of a thermoplastic material is heated and stretched to produce a parison 20 in which a non-heated portion of the tube 30 is formed as a thick portion 21 and a heated portion of the tube 30 is formed as a thin portion 22. A balloon 12 is formed from the parison 20, and the balloon 12 is disposed on a shaft 11 to produce the balloon catheter 10.

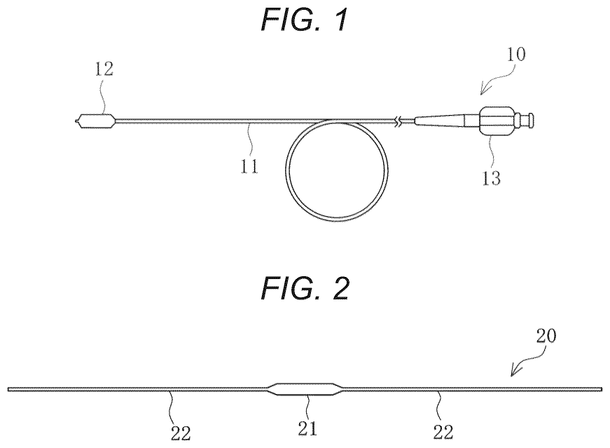

As shown in FIG. 1, the produced balloon catheter 10 includes the balloon 12 at a distal portion of the elongated shaft 11, and a hub 13 is provided on a proximal portion of the shaft 11. The balloon 12 can be inflated in a radial direction by an inflation fluid injected from the hub 13.

As shown in FIG. 2, the parison 20 extends along a major axis direction. The parison 20 includes the thick portion 21 having a large diameter in a central portion in the major axis direction, and thin portions 22 each having a diameter smaller than that of the thick portion 21 on both sides of the thick portion 21 in the major axis direction.

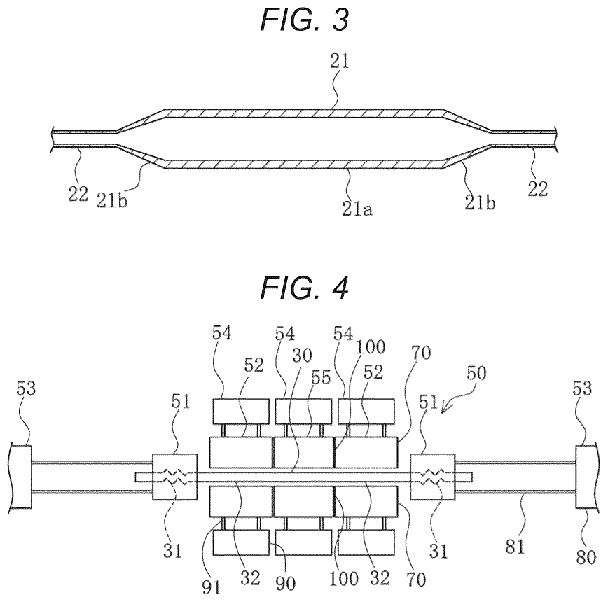

As shown in FIG. 3, the parison 20 has a hollow tubular shape. The thick portion 21 of the parison 20 includes a uniform-diameter portion 21a having the same diameter along the major axis direction, and inclined shoulder portions 21b located at both end portions of the uniform-diameter portion 21a in the major axis direction and each connecting the uniform-diameter portion 21a and the thin portion 22.

The parison 20 is formed of a thermoplastic material. In the present embodiment, the material for forming the parison 20 is a polyamide. The material for forming the parison 20 is not limited to a polyamide, and for example, polyolefins such as polyethylene, polypropylene, polybutene, an ethylene-propylene copolymer, an ethylene-vinyl acetate copolymer, an ionomer, or a mixture of two or more kinds of polyolefins, or a thermoplastic resin such as a soft polyvinyl chloride resin, a polyamide elastomer, a polyester, a polyester elastomer, a polyurethane, or a fluororesin can be used.

A production device 50 and a production method for the parison 20 will be described. As shown in FIG. 4, the production device 50 for the parison 20 includes fixing mechanisms 51 for fixing the tube 30, heating mechanisms 52 for heating a part of the tube 30, a cooling mechanism 55 for cooling another part of the tube 30, a pulling mechanism 53 for pulling the tube 30 along the major axis direction by moving the fixing mechanisms 51, and movement mechanisms 54 for opening and closing the heating mechanisms 52 and the cooling mechanism 55.

The fixing mechanisms 51 fix a pair of fixing portions 31, 31, which are separated from each other in the major axis direction, of the tube 30 extending along the major axis direction. The fixing mechanisms 51 each have a through space through which both end portions of the tube 30 can be inserted, and includes a fixing unit for pressing and holding the inserted tube 30 in the radial direction.

The heating mechanisms 52 can heat a pair of heating portions 32, 32, which are located between the pair of fixing portions 31, 31 of the tube 30 and separated from each other in the major axis direction. The heating mechanism 52 includes a pair of heating molds 70, 70, which are heat sources and are disposed on both sides of the tube 30 in the radial direction. The heating molds 70, 70 can be opened and closed by bringing the heating molds 70, 70 close to and separated (i.e., away) from each other along the radial direction of the tube 30 by the movement mechanism 54. In FIG. 4, the heating molds 70, 70 are separated from each other and are in an opened state in which the heating portions 32, 32 of the tube 30 are exposed.

The cooling mechanism 55 can cool at least a part of a portion between the pair of heating portions 32, 32 of the tube 30. The cooling mechanism 55 includes a pair of cooling molds 100, 100 which are cooling units and are disposed on both sides of the tube 30 in the radial direction. The cooling molds 100, 100 can be opened and closed by bringing the cooling molds 100, 100 close to and separated (i.e., away) from each other along the radial direction of the tube 30 by the movement mechanism 54. In FIG. 4, the cooling molds 100, 100 are in an opened state.

The pulling mechanism 53 includes an arm portion 81 interlocked with the fixing mechanism 51, and a main body portion 80 extending and contracting the arm portion 81. When the arm portion 81 is extended and contracted by the main body portion 80, the fixing mechanism 51 can be moved along the major axis direction of the tube 30, and the tube 30 fixed to the fixing mechanism 51 can be pulled along the major axis direction.

The movement mechanism 54 includes an arm portion 91 interlocked with the heating mold 70 or the cooling mold 100, and a main body portion 90 extending and contracting the arm portion 91. When the main body portion 90 is extended and contracted by the arm portion 91, the heating molds 70, 70 facing each other and the cooling molds 100, 100 facing each other can be moved to open and close.

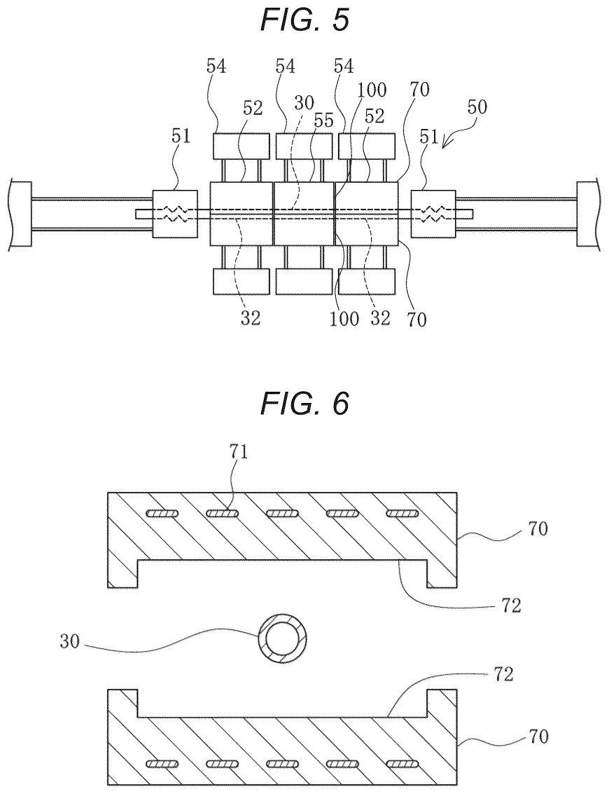

In order to produce the parison 20, first, as shown in FIG. 4, the fixing portions 31, 31 of the tube 30 are fixed to the fixing mechanisms 51, 51 of the production device 50, respectively (fixing step). Next, as shown in FIG. 5, the heating molds 70, 70, which face each other, of the heating mechanisms 52, 52 are brought close to each other to reach a closed state by the movement mechanisms 54, and the pair of heating portions 32, 32 of the tube 30 are covered and heated (heating step). The cooling molds 100, 100 of the cooling mechanism 55 are brought close to each other by the movement mechanism 54 to cover the portion between the pair of heating portions 32, 32 of the tube 30 and reach a closed state, and the portion between the pair of heating portions 32, 32 is cooled during the heating step (cooling step). The timing at which the cooling molds 100, 100 are closed to start cooling may be the same as the timing at which the heating molds 70, 70 of the heating mechanisms 52 are closed to start heating, or some time difference may be present, for example, the cooling molds 100, 100 are closed to start cooling before the heating molds 70, 70 are closed. The cooling step is continued while the heating step is performed.

As shown in FIG. 6, the pair of heating molds 70, 70 have concave wall surfaces 72, 72 on surfaces facing each other. The heating mold 70 includes heating units 71 in the heating mold 70. A heater can be used as the heating unit 71. In this case, the heating unit 71 may be provided on an outer surface of the heating mold 70 or may be a heating unit other than the heater. The heating mold 70 is heated by the heating units 71, and a temperature of the wall surface 72 can be raised.

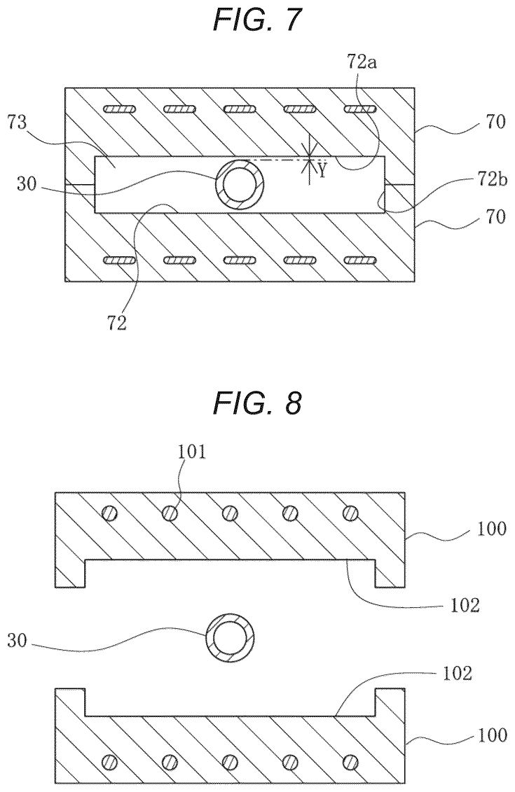

As shown in FIG. 7, the pair of heating molds 70, 70 are brought close to each other to reach a closed state, so that the tube 30 is accommodated in the pair of heating molds 70,70. The heating molds 70, 70 in the closed state have a space 73 surrounded by the wall surfaces 72, 72 having a rectangular cross-sectional shape obtained by cutting along a plane perpendicular to the major axis direction. The wall surfaces 72, 72 each have a long wall 72a extending in a horizontal direction and a short wall 72b extending in a vertical direction, which is a direction different from that of the long wall 72a, with a length smaller than that of the long wall 72a in a cross-sectional shape obtained by cutting along a plane perpendicular to the major axis direction. The tube 30 disposed in the space 73 formed by the pair of heating molds 70, 70 is close to the long walls 72a among the wall surfaces 72, 72. A minimum distance Y between the wall surface 72 and the tube 30 is a distance between the long wall 72a and the tube 30, and 0<Y≤1.5 mm is satisfied.

When the temperature of the wall surfaces 72, 72 is raised by the heating units 71, radiant heat from the wall surfaces 72, 72 and convection heat transfer in the space 73 heated by the wall surfaces 72, 72 are generated, and the tube 30 can be heated. Heating for the tube 30 is maintained for a certain period of time. In the heating step, the heating molds 70, 70 serving as a heat source maintain surfaces of the heating portions 32, 32 at a temperature higher than the glass transition temperature of the tube 30. In the present embodiment, the heating mold 70 is heated, for example, to a temperature of 80° C. or higher and 160° C. or lower (i.e., 80° C. to 160° C.) in the heating step. The heating step is performed, for example, for 1 second or longer and 30 seconds or shorter (i.e., 1 second to 30 seconds).

The wall surfaces 72, 72 of the heating molds 70, 70 are close to the tube 30, and therefore, the tube 30 can be stably heated by combining the radiant heat and the convection heat transfer. Therefore, even if the heating temperature of the heating molds 70, 70 is kept low, the heating for the tube 30 can be efficiently performed. In the heating step, by lowering the heating temperature of the heating molds 70, 70, a portion other than the heating portions 32, 32 of the tube 30 can be prevented from being heated, and therefore, the temperature rise in the portion can be prevented. Accordingly, a length of the parison 20 to be produced can be stably formed. A shape of the shoulder portion 21b of the parison 20 can be clearly formed, and therefore, it is relatively easy to identify the shape during inspection. When the shape of the shoulder portion 21b of the parison 20 is clear, it can be determined that the thick portion 21 of the parison 20 is not stretched. From the above, the parison 20 can be stably produced.

In the present embodiment, the production device 50 using the heating mold 70 shown in FIGS. 6 and 7 is described, and a heating mold according to the related art may be used.

As shown in FIG. 8, the pair of cooling molds 100, 100 have concave wall surfaces 102, 102 on surfaces facing each other. The cooling mold 100 includes cooling units 101 in the cooling mold 100, and the cooling units 101 can lower the temperature of the wall surfaces 102 to cool the tube 30 in a space 103. The cooling unit 101 may be a heat transfer tube through which cooling water flows. In this case, the cooling unit 101 is not limited to the heat transfer tube as long as it is a unit capable of cooling the cooling mold 100.

As shown in FIG. 9, the cooling molds 100, 100 facing each other are brought close to each other to form the space 103 surrounded by the wall surfaces 102, 102. The space 103 has a rectangular cross-sectional shape obtained by cutting along a plane perpendicular to the major axis direction, and can accommodate the tube 30.

The wall surfaces 102, 102 in a state in which the pair of cooling molds 100, 100 are closed, each have a long wall 102a extending in the horizontal direction and a short wall 102b extending in the vertical direction, which is a direction different from that of the long wall 102a, with a length smaller than that of the long wall 102a in a cross-sectional shape obtained by cutting along a plane perpendicular to the major axis direction. The tube 30 disposed in the space 103 formed by the pair of cooling molds 100, 100 is close to the long walls 102a, 102a of the wall surfaces 102, 102.

When the wall surfaces 102, 102 are cooled by the cooling units 101, radiation cooling to the wall surfaces 102, 102 and convection heat transfer in the space 103 cooled by the wall surfaces 102, 102 are generated, and the tube 30 can be cooled.

In the cooling step, a surface of the portion between the pair of heating portions 32, 32 of the tube 30 is maintained at a temperature lower than the glass transition temperature of the tube 30. In the cooling step, the time from a start of cooling the tube 30 to a state in which a temperature lower than the glass transition temperature of the tube 30 is maintained, for example, for 5 seconds or longer and 45 seconds or shorter (5 seconds to 45 seconds). A difference between a surface temperature of the tube 30 in the heating step and the surface temperature of the tube 30 in the cooling step can be, for example, 50° C. or higher and 140° C. or lower (50° C. to 140° C.). With the cooling step, a portion to be stretched in the stretching step is heated while the portion of the tube 30 to be the thick portion 21 is cooled, and a clear temperature difference can be established between the two portions. Accordingly, the temperature rise in the part of the tube 30 to be the thick portion 21 can be prevented, the shape of the shoulder portion 21b of the parison 20 to be produced can be clearly formed, and the parison 20 can be stably produced.

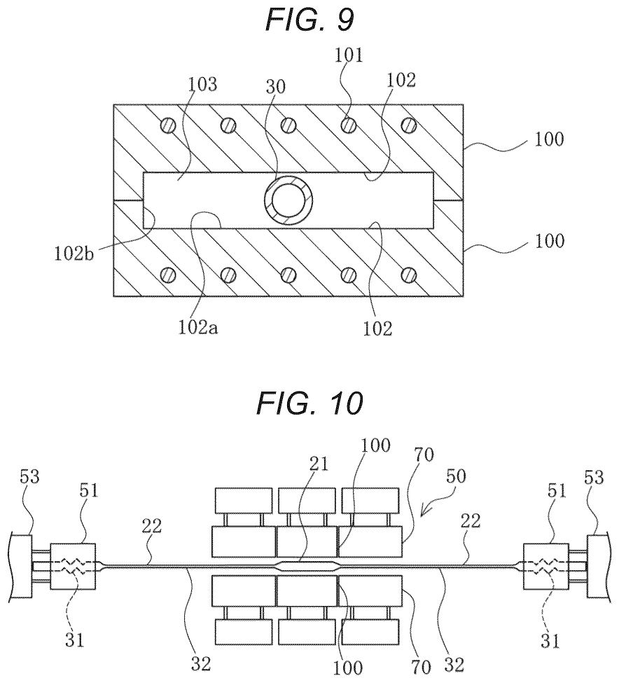

After the heating portions 32, 32 of the tube 30 are heated in the heating step while cooling a part of the tube 30 in the cooling step, the heating and cooling for the tube 30 are stopped, and as shown in FIG. 10, the tube 30 is pulled by the pulling mechanism 53 in a direction in which the pair of fixing portions 31, 31 are separated from each other, and the pair of heating portions 32, 32 of the tube 30 that are heated by the heating step are stretched (stretching step). During the stretching step, a step of separating the heating molds 70, 70 and the cooling molds 100, 100 from the surface of the tube 30 is performed by opening the heating molds 70, 70 and the cooling molds 100, 100 by the movement mechanisms 54. The heating molds 70, 70 and the cooling molds 100, 100 may be opened simultaneously with the start of the stretching step after the heating step, or may be opened during the stretching step. Note that, when the cooling step is performed by the cooling molds 100, 100 in which the wall surfaces 102, 102 are close to the surface of the tube 30, the stretching step may be performed while the cooling molds 100, 100 are closed after the cooling step.

With the stretching step, the heating portions 32, 32 of the tube 30 are stretched in the major axis direction, and the stretched portions are oriented to increase the strength. With the stretching step, the thick portion 21 having an outer diameter larger than that of the thin portions 22, 22 is formed between the pair of heating portions 32, 32 stretched to form the thin portions 22, 22 in the tube 30.

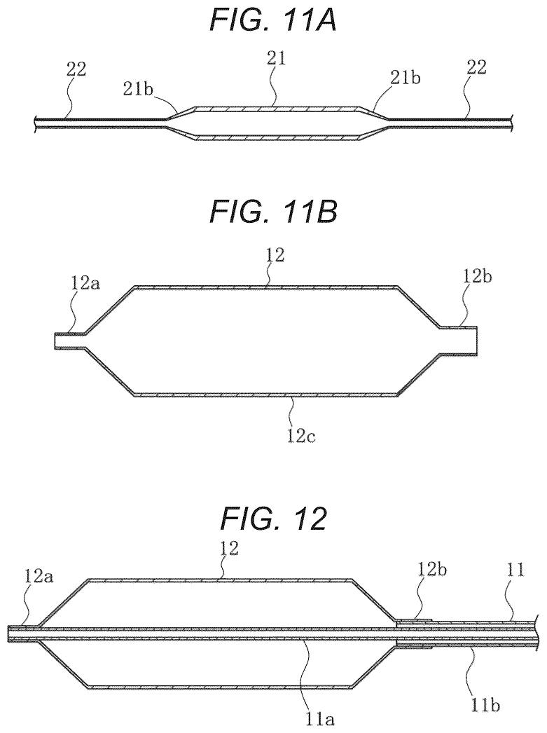

The balloon 12 is formed by biaxially stretching the thick portion 21 of the parison 20 shown in FIG. 11A and expanding the thick portion 21 as shown in FIG. 11B. The biaxial stretching process of the thick portion 21 is performed by blow molding such that the thick portion 21 is expanded in the biaxial directions, that is, the major axis direction and the radial direction. In the biaxial stretching process, only the thick portion 21, which is not stretched, of the tube 30 is expanded to form an intermediate portion 12c, and a distal portion 12a and a proximal portion 12b that extend forward and backward of the intermediate portion 12c. After the parison 20 is biaxially stretched, both end sides in the major axis direction are cut to remain portions with a predetermined length as the distal portion 12a and the proximal portion 12b, and the balloon 12 can be formed. As the details of the biaxial stretching process, known methods can be appropriately adopted. The intermediate portion 12c of the balloon 12 has a function of widening a stenosed site or the like during inflation. The distal portion 12a and the proximal portion 12b are liquid-tightly joined to the shaft 11 as described below.

After the balloon 12 is formed, the shaft 11 including the hub 13 is disposed in the formed balloon 12. Specifically, as shown in FIG. 12, an inner tubular shaft 11a is fixed to the distal portion 12a of the balloon 12 by a known method such as fusion bonding, an outer tubular shaft 11b disposed outside the inner tubular shaft 11a is fixed to the proximal portion 12b of the balloon 12 by the same method, and the shaft 11 is disposed in the balloon 12, thereby producing the balloon catheter 10.

In the method for producing a parison for a balloon catheter according to the present embodiment, a plurality of parisons 20 can also be produced by processing a plurality of tubes 30 at one time. As shown in FIG. 13, the plurality of tubes 30 can be arranged along the long walls 72a of the wall surfaces 72 in the space 73 of the heating molds 70, 70 of the heating mechanisms 52. The plurality of tubes 30 arranged in the space 73 are heated at one time by the heating molds 70, 70.

In the case where one or the plurality of tubes 30 are disposed in the space 73 of the heating molds 70, 70, a ratio of a cross-sectional area of the space 73 cut along a plane perpendicular to the major axis direction of the tube 30 to a total cross-sectional area of the tubes 30 cut along the plane perpendicular to the major axis direction of the tube 30 can be, for example, 2 or more and 10 or less (2 to 10), and preferably 4 or more and 8 or less (4 to 8).

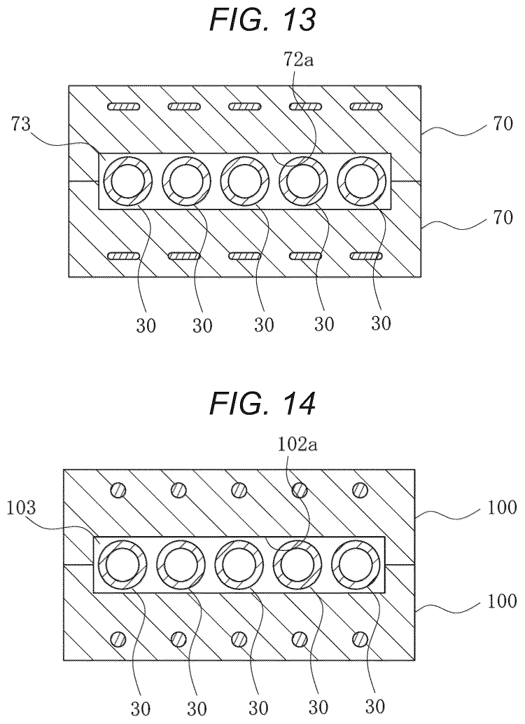

As shown in FIG. 14, the plurality of tubes 30 can be arranged along the long walls 102a of the wall surfaces 102 in the space 103 of the cooling molds 100, 100 of the cooling mechanism 55. The plurality of tubes 30 arranged in the space 103 are heated at one time by the cooling molds 100, 100.

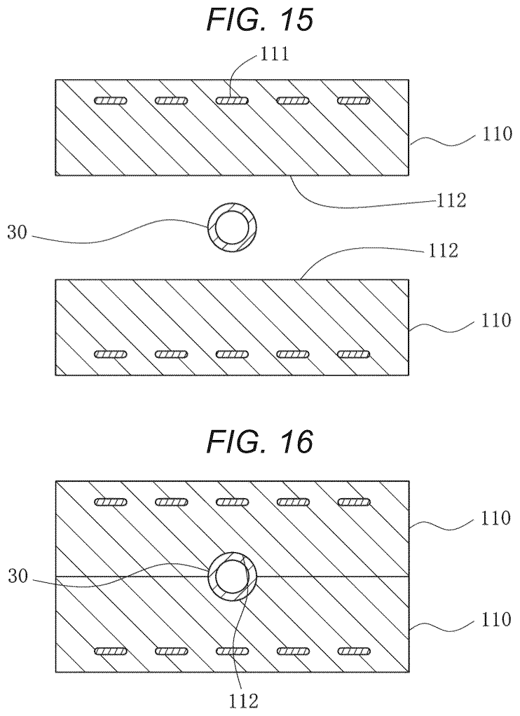

Next, a heating mold 110 and a cooling mold 120 according to a modification will be described. As shown in FIG. 15, the heating mold 110 includes heating units 111, and surfaces of the heating molds 110, 110 facing each other are planar wall surfaces 112. The heating mold 110 is formed of an elastic material.

As shown in FIG. 16, when the heating molds 110, 110 are closed, the wall surfaces 112 are deformed by the tube 30. Therefore, in the heating step, the heating molds 110, 110 serving as heat sources can be brought into contact with the entire circumference of a surface of the tube 30. When the heating molds 110, 110 are brought into contact with the surface of the tube 30 to perform heating, heat is transferred from the heating molds 110, 110 to the tube 30 by heat conduction, and the heating for the tube 30 can be efficiently performed even if the heating temperature of the heating molds 110, 110 is kept low.

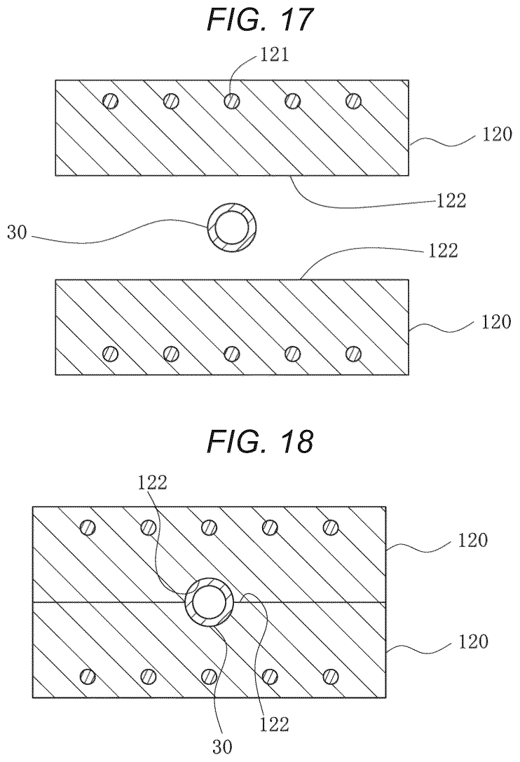

As shown in FIGS. 17 and 18, the cooling molds 120, 120 may be formed of an elastically deformable material and may be in contact with the surface of the tube 30. The cooling mold 120 including cooling units 121 has a wall surface 122 on a surface facing the opposing cooling mold 120, and is elastically deformed by the surface of the tube 30, so that the wall surfaces 122 come into contact with the entire circumference of the surface of the tube 30 when the cooling molds 120, 120 are closed. When the wall surfaces 122 of the cooling molds 120, 120 come into contact with the surface of the tube 30, heat is conducted between the cooling molds 120, 120 and the tube 30 by heat conduction, and the tube 30 can be efficiently cooled.

After the heating step and the cooling step are performed in a state in which the heating molds 110, 110 and the cooling molds 120, 120 are closed, the heating molds 110, 110 and the cooling molds 120, 120 are opened to be separated from the surface of the tube 30, and then the stretching step is performed.

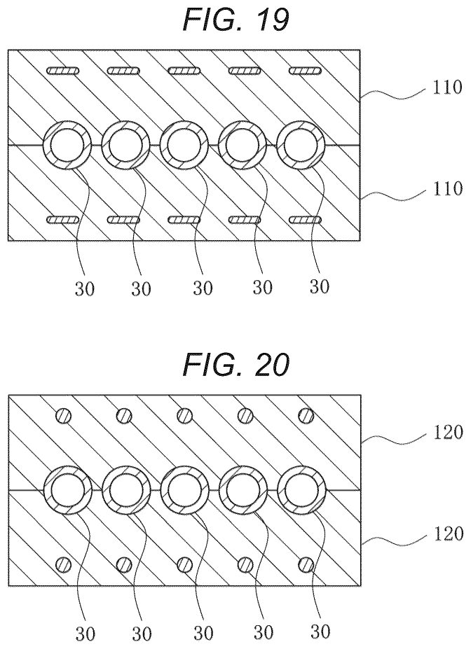

As shown in FIG. 19, the plurality of tubes 30 can be heated at one time by the heating molds 110, 100 in contact with the surfaces of the tubes 30. As shown in FIG. 20, the plurality of tubes 30 can be cooled at one time by the cooling molds 120, 120 in contact with the surfaces of the tubes 30.

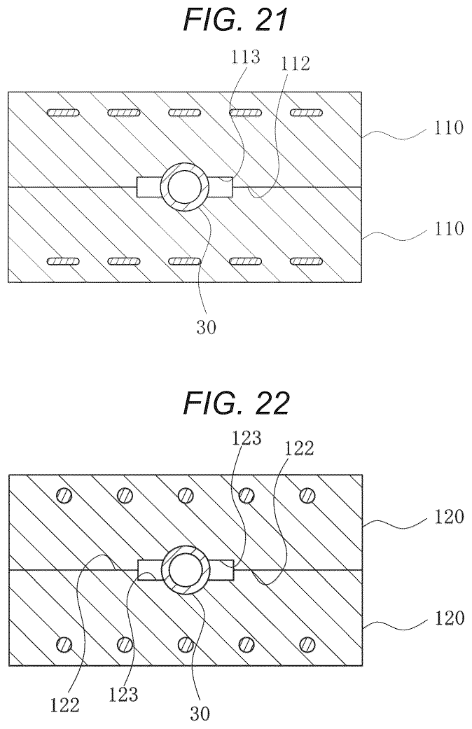

As shown in FIG. 21, the heating molds 110, 110 may each have a concave portion 113 in the wall surface 112, and the heating molds 110, 110 may come into contact with a part of a surface of the tube 30 in a circumferential direction. In this case, it is preferable that a contact area between the heating mold 110 and the surface of the tube 30 is larger than a non-contact area between the heating mold 110 and the surface of the tube 30. As shown in FIG. 22, the cooling molds 120, 120 may each have a concave portion 123 in the wall surface 122, and the cooling molds 120, 120 may come into contact with a part of the surface of the tube 30 in the circumferential direction. In this case, it is preferable that a contact area between the cooling mold 120 and the surface of the tube 30 is larger than a non-contact area between the heating mold 110 and the surface of the tube 30.

As described above, (1) a method for producing a parison 20 for a balloon catheter according to the present embodiment includes: fixing a pair of fixing portions 31, 31 of a thermoplastic tube 30 extending along a major axis direction, the pair of fixing portions 31, 31 being separated from each other in the major axis direction (a fixing step); heating a pair of heating portions 32, 32 of the tube 30, the pair of heating portions 32, 32 being located between the pair of fixing portions 31, 31 and separated from each other in the major axis direction (a heating step); cooling at least a part of a portion between the pair of heating portions 32, 32 of the tube 30 during the heating step; and pulling the tube 30 in a direction in which the pair of fixing portions 31, 31 are separated from each other, and stretching the pair of heating portions 32, 32 of the tube 30 heated by the heating step (a stretching step). A thick portion 21 having an outer diameter larger than that of the stretched portion is formed between the pair of heating portions 32, 32 stretched by the stretching step. In the method for producing the parison 20 for a balloon catheter configured as described above, when the tube 30 is heated, a clear temperature difference can be established between the heating portion and the portion of the tube 30 between the heating portions 32, 32. Therefore, the shape of the parison 20 can be stably formed in the stretching step.

(2) In the method for producing the parison 20 for a balloon catheter according to the above (1), in the cooling step, at least a part of a cooling unit may come into contact with a surface of the tube 30. Accordingly, the tube 30 can be stably cooled by heat conduction.

(3) In the method for producing the parison 20 for a balloon catheter according to the above (1), in the cooling step, a cooling unit may cool a space 103 surrounded by a wall surface 102 having a rectangular cross-sectional shape obtained by cutting along a plane perpendicular to the major axis direction. Accordingly, the tube 30 can be stably cooled by combining the radiant heat and the convection heat transfer.

(4) In the method for producing the parison 20 for a balloon catheter according to any one of the above (1) to (3), in the cooling step, a surface of the portion between the pair of heating portions 32, 32 may be maintained at a temperature lower than a glass transition temperature of the tube 30. Accordingly, the temperature rise in the part of the tube 30 to be the thick portion 21 can be prevented, and the shape of the shoulder portion of the parison 20 to be produced can be clearly formed.

(5) In the method for producing the parison 20 for a balloon catheter according to any one of the above (1) to (4), in the heating step, a surface of the heating portion may be maintained at a temperature higher than the glass transition temperature of the tube 30. Accordingly, the heating portion of the tube 30 can be reliably stretched in the stretching step to increase the strength.

(6) In the method for producing the parison 20 for a balloon catheter according to any one of the above (1) to (5), a difference between a surface temperature of the tube 30 in the heating step and a surface temperature of the tube 30 in the cooling step may be 50° C. or higher and 140° C. or lower. Accordingly, the shape of the thick portion 21 of the tube 30 can be stably formed by providing a sufficient temperature difference between the heating portion and the cooling portion of the tube 30.

(7) In the method for producing the parison 20 for a balloon catheter according to any one of the above (1) to (6), in the cooling step, a time from a start of cooling the tube 30 to a state in which a temperature lower than the glass transition temperature of the tube 30 is maintained may be 5 seconds or longer and 45 seconds or shorter. Accordingly, the non-heated portion of the tube 30 can be reliably cooled in accordance with the heating time of the tube 30.

(8) A method for producing the balloon catheter 10 according to the present embodiment includes: biaxially stretching the parison 20 for a balloon catheter, which is produced by the method according to any one of the above (1) to (7), to form a balloon 12; and disposing a shaft 11 in the formed balloon 12. The shape of the parison 20 for a balloon catheter is stably formed by the production method according to any one of the above (1) to (7), and therefore, the balloon catheter 10 including the balloon 12 having a stable shape can be produced.

(9) A production device 50 for the parison 20 for a balloon catheter according to the present embodiment includes: a fixing mechanism 51 configured to fix a pair of fixing portions 31, 31 of a thermoplastic tube 30 extending along a major axis direction, the pair of fixing portions 31, 31 being separated from each other in the major axis direction; a heating mechanism 52 configured to heat a pair of heating portions 32, 32 of the tube 30, the pair of heating portions 32, 32 being located between the pair of fixing portions 31, 31 and separated from each other in the major axis direction; the cooling mechanism 55 configured to cool a portion between the pair of heating portions 32, 32 of the tube 30; and a pulling mechanism 53 configured to move the fixing mechanism 51 in a direction in which a distance between the pair of fixing portions 31, 31 of the tube 30 is increased. In the device for producing the parison 20 for a balloon catheter configured as described above, when the tube 30 is heated, a clear temperature difference can be established between the heating portion and the portion of the tube 30 between the heating portions 32, 32, and the shape of the parison 20 can be stably formed.

The detailed description above describes embodiments of a method for producing a parison which is an intermediate product during production of a balloon provided in a balloon catheter, a method for producing a balloon catheter, and a device for producing a parison for a balloon catheter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A method for producing a parison for a balloon catheter, the method comprising:
fixing a pair of fixing portions of a thermoplastic tube extending along a major axis direction, the pair of fixing portions being separated from each other in the major axis direction;
heating a pair of heating portions of the tube, the pair of heating portions being located between the pair of fixing portions and separated from each other in the major axis direction;
cooling at least a part of a portion between the pair of heating portions of the tube during the heating of the pair of heating portions of the tube; and
pulling the tube in a direction in which the pair of fixing portions are separated from each other, and stretching the pair of heating portions of the tube heated by the heating of the pair of heating portions of the tube, wherein
a thick portion having an outer diameter larger than that of the stretched portion is formed between the pair of heating portions stretched by the pulling of the tube in the direction in which the pair of fixing portions are separated from each other.

2. The method for producing a parison for a balloon catheter according to claim 1, wherein
in the cooling of at least the part of the portion between the pair of heating portions of the tube, at least a part of a cooling unit comes into contact with a surface of the tube.

3. The method for producing a parison for a balloon catheter according to claim 1, wherein
in the cooling of at least the part of the portion between the pair of heating portions of the tube, a cooling unit cools a space surrounded by a wall surface having a rectangular cross-sectional shape obtained by cutting along a plane perpendicular to the major axis direction.

4. The method for producing a parison for a balloon catheter according to claim 1, wherein
in the cooling of at least the part of the portion between the pair of heating portions of the tube, a surface of the portion between the pair of heating portions is maintained at a temperature lower than a glass transition temperature of the tube.

5. The method for producing a parison for a balloon catheter according to claim 4, wherein
in the heating of the pair of heating portions of the tube, a surface of the heating portion is maintained at a temperature higher than the glass transition temperature of the tube.

6. The method for producing a parison for a balloon catheter according to claim 1, wherein
a difference between a surface temperature of the tube in the heating of the pair of heating portions of the tube and a surface temperature of the tube in the cooling of at least the part of the portion between the pair of heating portions of the tube is 50° C. to 140° C.

7. The method for producing a parison for a balloon catheter according to claim 4, wherein
in the cooling of at least the part of the portion between the pair of heating portions of the tube, a time from a start of cooling the tube to a state in which a temperature lower than the glass transition temperature of the tube is maintained for 5 seconds to 45 seconds.

8. A method for producing a balloon catheter, the method comprising:
biaxially stretching the parison for a balloon catheter, which is produced by the method according to claim 1, to form a balloon; and
disposing a shaft in the formed balloon.

9. A method for producing a parison for a balloon catheter, the method comprising:
fixing a pair of fixing portions of a tube extending along a major axis direction, the pair of fixing portions being separated from each other in the major axis direction;
heating a pair of heating portions of the tube, the pair of heating portions being located between the pair of fixing portions and separated from each other in the major axis direction;
cooling at least a part of a portion between the pair of heating portions of the tube during the heating of the pair of heating portions of the tube; and
pulling the tube in a direction in which the pair of fixing portions are separated from each other, and stretching the pair of heating portions of the tube heated by the heating of the pair of heating portions of the tube.

10. The method for producing a parison for a balloon catheter according to claim 9, wherein in the cooling of at least the part of the portion between the pair of heating portions of the tube, further comprises:
contacting at least a part of a cooling unit on a surface of the tube.

11. The method for producing a parison for a balloon catheter according to claim 9, wherein in the cooling of at least the part of the portion between the pair of heating portions of the tube, further comprises:
cooling a space surrounded by a wall surface having a rectangular cross-sectional shape obtained by cutting along a plane perpendicular to the major axis direction with a cooling unit.

12. The method for producing a parison for a balloon catheter according to claim 9, wherein in the cooling of at least the part of the portion between the pair of heating portions of the tube, further comprises:
maintaining a surface of the portion between the pair of heating portions at a temperature lower than a transition temperature of the tube; and
maintaining a surface of the heating portion at a temperature higher than the transition temperature of the tube.

13. The method for producing a parison for a balloon catheter according to claim 9, further comprising:
establishing a difference between a surface temperature of the tube in the heating of the pair of heating portions of the tube and a surface temperature of the tube in the cooling of at least the part of the portion between the pair of heating portions of the tube of 50° C. to 140° C.

14. The method for producing a parison for a balloon catheter according to claim 12, wherein in the cooling of at least the part of the portion between the pair of heating portions of the tube, further comprises:
maintaining a time from a start of cooling the tube to a state in which a temperature lower than the glass transition temperature of the tube for 5 seconds to 45 seconds.

15. A device for producing a parison for a balloon catheter, the device comprising:
a fixing mechanism configured to fix a pair of fixing portions of a thermoplastic tube extending along a major axis direction, the pair of fixing portions being separated from each other in the major axis direction;
a heating mechanism configured to heat a pair of heating portions of the tube, the pair of heating portions being located between the pair of fixing portions and separated from each other in the major axis direction;
a cooling mechanism configured to cool a portion between the pair of heating portions of the tube; and
a pulling mechanism configured to move the fixing mechanism in a direction in which a distance between the pair of fixing portions of the tube is increased.

16. The device for producing the parison for the balloon catheter according to claim 15, further comprising:
a cooling unit; and
wherein at least a part of the cooling unit is configured to come into contact with a surface of the tube in the cooling of at least the part of the portion between the pair of heating portions of the tube.

17. The device for producing the parison for the balloon catheter according to claim 15, further comprising
a cooling unit; and
wherein the cooling unit is configured to cool a space surrounded by a wall surface having a rectangular cross-sectional shape obtained by cutting along a plane perpendicular to the major axis direction wherein in the cooling of at least the part of the portion between the pair of heating portions of the tube.

18. The device for producing the parison for the balloon catheter according to claim 15, wherein in the cooling of at least the part of the portion between the pair of heating portions of the tube, a surface of the portion between the pair of heating portions is configured to be maintained by the cooling mechanism at a temperature lower than a transition temperature of the tube.

19. The device for producing the parison for the balloon catheter according to claim 18, wherein in the heating of the pair of heating portions of the tube, a surface of the heating portion is configured to be maintained by the heating mechanism at a temperature higher than the glass transition temperature of the tube.

20. The device for producing the parison for the balloon catheter according to claim 15, wherein the heating mechanism and the cooling mechanism are configured to maintain a difference between a surface temperature of the tube in the heating of the pair of heating portions of the tube and a surface temperature of the tube in the cooling of at least the part of the portion between the pair of heating portions of the tube is 50° C. to 140° C.
